(19) United States
(12) Patent Application Publication
(10) Pub. No.: US 2014/0364417 A1
(43) Pub. Date: Dec. 11, 2014

(54) METHOD OF TREATING ATRIAL FIBRILLATION

(71) Applicants: Gilead Sciences, Inc., Foster City, CA (US); Beth Israel Deaconess Medical Center, Inc., Boston, MA (US)

(72) Inventors: Luiz Belardinelli, Palo Alto, CA (US); Richard Verrier, Wellesley, MA (US); Dewan Zeng, Palo Alto, CA (US)

(21) Appl. No.: 14/269,026

(22) Filed: May 2, 2014

Related U.S. Application Data
(60) Provisional application No. 61/819,477, filed on May 3, 2013.

(51) Int. Cl. A61K 31/55; A61K 31/496

(52) U.S. Cl. CPC A61K 31/55; A61K 31/496
USPC 514/212.07; 514/252.12

(57) ABSTRACT

The present disclosure relates to a method for the treatment or prevention of atrial fibrillation and/or atrial flutter comprising coadministration of a therapeutically effective amount of ivabradine or a pharmaceutically acceptable salt thereof and a therapeutically effective amount of ranolazine. Also provided are methods for modulating ventricular and atrial rate. This disclosure also relates to pharmaceutical formulations that are suitable for such combined administration.

### CROSS REFERENCE TO RELATED APPLICATIONS

This application claims the benefit under 35 U.S.C. 119(e) to U.S. Provisional Application Ser. No. 61/819,477, filed on May 3, 2013, the entirety of which is incorporated herein by reference.

### FIELD

The present disclosure relates to methods of treating and/or preventing atrial fibrillation and/or atrial flutter by coadministration of therapeutically effective amounts of ranolazine and ivabradine or pharmaceutically acceptable salts thereof. This disclosure also relates to pharmaceutical formulations that are suitable for such coadministration.

### BACKGROUND

Atrial fibrillation (AF) is the most prevalent arrhythmia, the incidence of which increases with age. It is estimated that 8% of all people over the age of 80 experience this type of abnormal heart rhythm and AF accounts for one-third of hospital admissions for cardiac rhythm disturbances. Over 2.2 million people are believed to have AF in the Unites States alone (Fuster et al Circulation 2006 114 (7): e257-354). Although atrial fibrillation is often asymptomatic it may cause palpitations or chest pain. Prolonged atrial fibrillation often results in the development of congestive heart failure and/or stroke. Heart failure develops as the heart attempts to compensate for the reduced cardiac efficiency while stroke may occur when thrombi form in the atria, pass into the blood stream and lodge in the brain. Pulmonary emboli may also develop in this manner.

Current methods for treating AF include electric and/or chemical cardioversion and radiofrequency ablation. In addition, in patients in whom sinus rhythm (rhythm control) cannot be achieved, reduction of ventricular rate (rate control) is an alternative strategy to treat atrial fibrillation. Anticoagulants, such as warfarin, dabigatran, and heparin, are typically prescribed in order to avoid stroke. While there is currently some debate regarding the choice between rate and rhythm control (Roy et al. N Engl J Med 2008 358:25; 2667-2677) rate control is typically achieved by the use of beta blockers or calcium channel blockers. Contemporary pharmacologic approaches for controlling ventricular rate during atrial fibrillation (AF) involve slowing conduction through the atrioventricular (AV) node by administering “nodal” agents, which act on L-type calcium channels or on beta-adrenergic receptors. These rate control agents, such as β-blockers or calcium channel blockers, slow conduction through the AV node but depress the ventricular mechanical function because they are not selective for AV node. Further, these agents also reduce contractility, which can be particularly problematic in patients with heart failure, who have elevated risk of AF (Go et al. Circulation 2014; 129:e28-e292).

There is a need in the art for therapeutics that are more effective in treating atrial fibrillation without the undesirable side effects.

### SUMMARY

It has now been found that the combination of ivabradine and ranolazine has synergism resulting in potent electrophysiological actions leading to significant reduction in ventricular rate both by slowing A-V conduction and by decreasing dominant frequency of AF.

The present disclosure is based on the surprising and unexpected discovery that coadministration of ivabradine and ranolazine provides ventricular and/or atrial rate control. The ability to control the rate is useful for treating or preventing atrial fibrillation and/or atrial flutter in patients, as well as a variety of other cardiac conditions, which are described throughout. It is further contemplated that the coadministration is useful when ivabradine is administered in a therapeutically effective dose and ranolazine is administered in a therapeutically effective dose.

Accordingly, in one aspect, the disclosure is directed to a method for controlling ventricular rate during atrial fibrillation or atrial flutter in a human patient in need thereof, comprising administering to the patient a therapeutically effective amount of ivabradine or a pharmaceutically acceptable salt thereof and a therapeutically effective amount of ranolazine.

In another aspect, the disclosure is directed to a method for improving left ventricular function during atrial fibrillation or atrial flutter in a human patient in need thereof, comprising administering to the patient a therapeutically effective amount of ivabradine or a pharmaceutically acceptable salt thereof and a therapeutically effective amount of ranolazine.

In another aspect, the disclosure is directed to a method for treatment and/or prevention of atrial fibrillation or atrial flutter in a patient in need thereof. The method comprises coadministration of a therapeutically effective amount of ivabradine or pharmaceutically acceptable salt thereof and a therapeutically effective amount of ranolazine.

In another aspect, the disclosure is directed to a method for increasing the S-H interval (stimulus-to-His bundle interval) in a human patient in need thereof, comprising administering to the patient a therapeutically effective amount of ivabradine or a pharmaceutically acceptable salt thereof and a therapeutically effective amount of the ranolazine.

In another aspect, the disclosure is directed to a method for increasing the PR interval in a human patient in need thereof, comprising administering to the patient a therapeutically effective amount of ivabradine or a pharmaceutically acceptable salt thereof and a therapeutically effective amount of the ranolazine.

In another aspect, the disclosure is directed to a method for increasing the A-H interval in a human patient in need thereof, comprising administering to the patient a therapeutically effective amount of ivabradine or a pharmaceutically acceptable salt thereof and a therapeutically effective amount of the ranolazine.

In another aspect, the disclosure is directed to a method for modulating ventricular and/or atrial rate in a patient in need thereof, said method comprising administering to the patient a therapeutically effective amount of ranolazine and a therapeutically effective amount of ivabradine or a pharmaceutically acceptable salt thereof.

In another aspect, the disclosure is directed to a method for providing rate control of the ventricles and/or atria in a patient in need thereof, said method comprising administering to the patient a therapeutically effective amount of ranolazine and a therapeutically effective amount of ivabradine or a pharmaceutically acceptable salt thereof.

In another aspect, the disclosure is directed to a method for maintaining or improving cardiac function during atrial fibrillation in a human patient in need thereof, comprising administering to the patient a therapeutically effective amount of ivabradine or a pharmaceutically acceptable salt thereof and a therapeutically effective amount of ranolazine.

In another aspect, the disclosure is directed to a method for maintaining or improving cardiac mechanical function and/or arterial blood pressure during atrial fibrillation in a human patient in need thereof, comprising administering to the patient a therapeutically effective amount of ivabradine or a pharmaceutically acceptable salt thereof and a therapeutically effective amount of ranolazine.

In another aspect, the disclosure is directed to a method of decreasing dominant frequency of atrial fibrillation in a patient in need thereof, said method comprising administering to the patient a therapeutically effective amount of ranolazine and a therapeutically effective amount of ivabradine or a pharmaceutically acceptable salt thereof.

In another aspect, the disclosure is directed to a pharmaceutical formulation comprising a therapeutically effective amount of ivabradine or pharmaceutically acceptable salt thereof, a therapeutically effective amount of ranolazine, and a pharmaceutically acceptable carrier.

In another aspect of the disclosure a method is provided for the treatment of atrial fibrillation comprising the coadministration of a therapeutically effective amount of ivabradine or a pharmaceutically acceptable salt thereof and a therapeutically effective amount of ranolazine.

The ivabradine or a pharmaceutically acceptable salt thereof and ranolazine may be administered separately or together in separate or a combined dosage unit. If administered separately, the ranolazine may be administered before or after administration of the ivabradine but typically the ranolazine will be administered prior to the ivabradine. In another embodiment, ivabradine or a pharmaceutically acceptable salt thereof and ranolazine may be administered together in a fixed dose combination.

### BRIEF DESCRIPTION OF THE DRAWINGS

FIG. 1 illustrates the synergistic effect of ranolazine and ivabradine alone and in combination to increase the S-H (stimulus-to-His bundle) interval (AV nodal conduction). Ran: ranolazine (2 μM, n=2): Iva: ivabradine (0.1 μM, n=2); different from the Σ(R+I), a calculated sum of the experimentally measured individual effects of ranolazine and ivabradine; *, the experimentally measured value for the effect of the combination of ranolazine and ivabradine is significantly greater than the calculated Σ(R+I), indicating a synergistic effect rather than an additive effect.

FIG. 2 illustrates that coadministration of ranolazine and ivabradine show synergistic effects on Atrial-His (A-H) interval at pacing rate 180 beats/min (bpm). This interval represents the conduction time between the atrium and AV node as documented by signals from the atrium and His bundle electrograms. FIG. 2 demonstrates that the effects on A-H interval are more than additive. Specifically, ivabradine increases A-H interval by 31.2 msec (p=0.018). Ranolazine increases A-H interval by 18.4 msec (p=0.0095). Thus, their individual additive effects are 49.6 msec. The effect of the agents in combination is 73.6 msec (p=0.0026), which is unexpectedly greater than the contributions of the drugs given separately. These findings suggest that the combination has a greater effect in increasing A-H interval during a relatively high atrial pacing rate (180 bpm), as in atrial fibrillation. The combination of Iva and Ran significantly increases the A-H interval by 55.2 msec over administration of Ran alone (p=0.0091). These results are further described in Example 2. Similar synergistic effects are seen for pacing at 130 beats/min and for an ivabradine dose of 0.10 mg/kg at both pacing rates.

FIG. 3 illustrates that the combination of ranolazine and ivabradine synergistically act to maintain and/or improve arterial blood pressure during atrial fibrillation. Ranolazine given alone shows no significant improvement of arterial blood pressure during AF (Δ C ontrol/Ran=3.36 mmHg; p=0.36). However, the combination exerts a highly significant improvement (Δ Control/Iva+Ran=11.64 mmHg; p=0.0092). A Ran/Iva+Ran=8.33 mmHg; p=0.12. These results are further described in Example 2.

FIG. 4 illustrates that ivabradine alone shows a small, not significant decrease in arterial blood pressure during atrial fibrillation. These results are further described in Example 2.

FIG. 5, upper panel illustrates that during right atrial pacing at 180 bpm, both ivabradine (0.25 mg/kg, bolus) and ranolazine increased PR interval (p<0.02, p<0.01, respectively) and their combination further increased PR interval above the sum of the increases by each agent separately (p<0.01 for each drug). The net result was a significant increase in PR interval above baseline by combined administration of the agents (p<0.01). The lower panel illustrates that neither drug nor their combination altered QT interval. These results are further described in Example 2.

FIG. 6, left panel illustrates that during right atrial pacing at 180 beats/min, ivabradine (0.25 mg/kg, bolus) increased atrial-His (A-H) interval (p<0.01) but ranolazine did not (NS). The combination of agents increased A-H interval above the sum of the increases by each agent separately (p<0.03 compared to ivabradine, p<0.01 compared to ranolazine). The net result was a significant increase in A-H interval above baseline by combined administration of the agents (p<0.01). The right panel illustrates that neither drug nor their combination altered His-ventricular (H-V) interval. These results are further described in Example 2.

FIG. 7 illustrates the electrocardiographic changes in response to ranolazine alone (2.4 mg/kg, i.v., bolus followed by 0.135 mg/kg/min) and with subsequent ivabradine (0.25 mg/kg, i.v., bolus) administration during right atrial pacing at 180 beats/min in a representative experiment. Ranolazine alone resulted in a mild increase in A-H interval from 100 to 107 msec and a greater increase to 145 msec when combined with ivabradine. Ranolazine increased PR interval from 123 to 131 msec and to 162 msec when combined with ivabradine. HV intervals were unchanged by ranolazine alone (from 36 to 35 msec) or when combined with ivabradine (to 35 msec). QT intervals were also unchanged by ranolazine alone (from 283 to 282 msec) or when combined with ivabradine (to 279 msec). RA=right atrium; HBE=His bundle electrogram; RV=right ventricle. These results are further described in Example 2.

FIG. 8 illustrates that the combination of ivabradine (0.25 mg/kg, i.v., bolus) and ranolazine (2.4 mg/kg, i.v., bolus followed by 0.135 mg/kg/min continuous infusion) reduced dominant frequency of atrial fibrillation (p<0.01) although neither drug alone was effective. These results are further described in Example 2.

FIG. 9, top panel illustrates that ivabradine alone (0.25 mg/kg, i.v., bolus) lowered ventricular rate during atrial fibrillation (AF) (p<0.05) but ranolazine alone did not (NS). The combination of agents decreased ventricular rate below sum of the decreases by each agent separately (p≦0.014 compared to ivabradine, p<0.02 compared to ranolazine). The net result was a significant decrease in ventricular rate below baseline by combined administration of the agents (p=0.01). The bottom panel presents the simultaneous electrocardiograms obtained from right atrial and right ventricular electrode catheters from a representative experiment illustrating the decrease in ventricular rate during atrial fibrillation following administration of ivabradine (0.25 mg/kg, bolus) and ranolazine. These results are further described in Example 2.

### DETAILED DESCRIPTION

### 1. Definitions and General Parameters

As used in the present specification, the following words and phrases are generally intended to have the meanings as set forth below, except to the extent that the context in which they are used indicates otherwise.

It is to be noted that as used herein and in the claims, the singular forms “a,” “an” and “the” include plural referents unless the context clearly dictates otherwise. Thus, for example, reference to “a pharmaceutically acceptable carrier” in a composition includes two or more pharmaceutically acceptable carriers, and so forth.

“Comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

“Ivabradine”, “IVA”, or “Iva” refers to 3-{3-[{[(7S)-3,4-dimethoxybicyclo[4.2.0]octa-1,3,5-trien-7-yl]methyll}-(methyl)amino]propyl}-7,8-dimethoxy-1,3,4,5-tetrahydro-2H-3-benzapenin-2-one, or a pharmaceutically acceptable salt thereof, which has the following chemical formula:

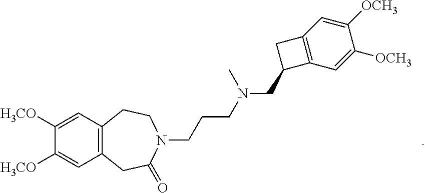 [SMILES: COc1cc2c(cc1OC)CC(=O)N(CCCN(C)C[C@H]1Cc3cc(OC)c(OC)cc31)CC2]

Ivabradine, its derivatives, and salts thereof, for example, ivabradine hydrochloride, have been described in U.S. Pat. No. 5,296,482.

“Ranolazine”, “RAN”, or “Ran” is described in U.S. Pat. No. 4,567,264 and refers to the chemical compound (±)-N-(2,6-dimethylphenyl)-4-[2-hydroxy-3-(2-methoxyphenoxy)-propyl]-1-piperazineacetamide, and its pharmaceutically acceptable salts. Ranolazine is represented by the formula:

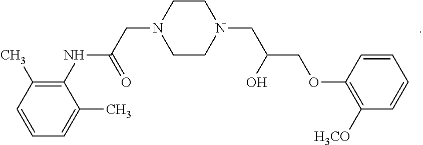 [SMILES: COc1ccccc1OCC(O)CN1CCN(CC(=O)Nc2c(C)cccc2C)CC1]

As used herein, the term “pharmaceutically acceptable salt” refers to a salt of a compound that is derived from a variety of physiologically acceptable organic and inorganic counter ions. Such counter ions are well known in the art and include, by way of example only, sodium, potassium, calcium, magnesium, aluminum, lithium and ammonium, for example tetraalkylammonium, and the like, when the molecule contains an acidic functionality; and salts of organic or inorganic acids, such as hydrochloride, sulfate, phosphate, diphosphate, nitrate hydrobromide, tartrate, mesylate, acetate, malate, maleate, fumarate, tartrate, succinate, citrate, lactate, pamoate, salicylate, stearate, methanesulfonate, p-toluenesulfonate, and oxalate, and the like, when the molecule contains a basic functionality. Suitable pharmaceutically acceptable salts also include those listed in Remington's Pharmaceutical Sciences, 17th Edition, pg. 1418 (1985) and P. Heinrich Stahl, Camille G. Wermuth (Eds.), Handbook of Pharmaceutical Salts Properties, Selection, and Use; 2002. Examples of acid addition salts include those formed from acids such as hydroiodic, phosphoric, metaphosphoric, nitric and sulfuric acids, and with organic acids, such as alginic, ascorbic, anthranilic, benzoic, camphorsulfuric, citric, embonic (pamoic), ethanesulfonic, formic, fumaric, furoic, galacturonic, gentisic, gluconic, glucuronic, glutamic, glycolic, isonicotinic, isothionic, lactic, malic, mandelic, methanesulfonic, mucic, pantothenic, phenylacetic, propionic, saccharic, salicylic, stearic, succinic, sulfinilic, trifluoroacetic and arylsulfonic for example benzenesulfonic and p-toluenesulfonic acids. Examples of base addition salts formed with alkali metals and alkaline earth metals and organic bases include chloroprocaine, choline, N,N-dibenzylethylenediamine, diethanolamine, ethylenediamine, lysine, meglumaine (N-methylglucamine), and procaine, as well as internally formed salts. Salts having a non-physiologically acceptable anion or cation are within the scope of the disclosure as useful intermediates for the preparation of physiologically acceptable salts and/or for use in non-therapeutic, for example, in vitro, situations.

The term “therapeutically effective amount” refers to the amount of a compound, such as ranolazine or ivabradine, sufficient to effect treatment, as defined below, when administered to a mammalian patient, such as a human patient, in need of such treatment. The therapeutically effective amount will vary depending upon the specific activity of the therapeutic agent being used, the severity of the patient's disease state, and the age, physical condition, existence of other disease states, and nutritional status of the patient. Additionally, other medications the patient may be receiving will affect the determination of the therapeutically effective amount of the therapeutic agent to administer. In some embodiments, the term “therapeutically effective amount” refers to a synergistically effective amount.

“Synergistic” means that the therapeutic effect of ivabradine when administered in combination with ranolazine (or vice-versa) is greater than the predicted additive therapeutic effects of ivabradine and ranolazine when administered alone. The term “synergistically effective amount” may refer to a subtherapeutic amount, meaning that the amount required for the desired effect is lower than when the drug is used alone.

The term “treatment” or “treating” means any treatment of a disease or condition in a subject, such as a mammal, including: 1) preventing or protecting against the disease or condition, that is, causing the clinical symptoms not to develop; 2) inhibiting the disease or condition, that is, arresting or suppressing the development of clinical symptoms; and/or 3) relieving the disease or condition that is, causing the regression of clinical symptoms.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

The term “susceptible” refers to a patient who has had at least one occurrence of the indicated condition.

The term “patient” typically refers to a “mammal” which includes, without limitation, human, monkeys, rabbits, mice, domestic animals, such as dogs and cats, farm animals, such as cows, horses, or pigs, and laboratory animals.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Atrial fibrillation” or “AF” occurs when the heart's two upper chambers (the right and left atria) quiver instead of beating and contracting rhythmically. Electrocardiographically, AF is characterized by a highly disorganized atrial electrical activity that often results in fast beating of the heart's two lower chambers (the right and left ventricles). Symptoms experienced by patients with AF include palpitation, fatigue, and dyspnea (shortness of breath).

There are three types of AF based on the presentation and duration of the arrhythmia: a) Paroxysmal AF: recurrent AF (>2 episodes) that starts and terminates spontaneously within 7 days (paroxysmal AF starts and stops spontaneously); b) Persistent AF: sustained AF that lasts longer than 7 days or requires termination by pharmacologic or electrical cardioversion (electrical shock); and c) Permanent AF: long standing AF (for >1 year duration) in which normal sinus rhythm cannot be maintained even after treatment, or when the patient and physician have decided to allow AF to continue without further efforts to restore sinus rhythm.

“Atrial flutter” is an abnormal heart rhythm that occurs in the atria of the heart. When it first occurs, it is usually associated with a fast heart rate or tachycardia (230-380 beats per minute (bpm)), and falls into the category of supra-ventricular tachycardias. While this rhythm occurs most often in individuals with cardiovascular disease (e.g. hypertension, coronary artery disease, and cardiomyopathy), it may occur spontaneously in people with otherwise normal hearts. It is typically not a stable rhythm, and frequently degenerates into atrial fibrillation (AF).

“AV conduction” or “atrioventricular conduction” is the forward conduction of the cardiac impulse from the atria to ventricles via the “atrioventricular node” or “AV node”, represented in an electrocardiogram by the P-R interval. The AV node is a part of electrical control system of the heart that electrically connects atrial and ventricular chambers and coordinates heart rate. The AV node is an area of specialized tissue between the atria and the ventricles of the heart, specifically in the posteroinferior region of the interatrial septum near the opening of the coronary sinus, which conducts the normal electrical impulse from the atria to the ventricles. “AV conduction” during normal cardiac rhythm occurs through two different pathways: the first has a slow conduction velocity but shorter refractory period, whereas the second has a faster conduction velocity but longer refractory period.

The term “modulate” means to increase, decrease, or otherwise provide control.

“Modulating ventricular and/or atrial rate” has been shown to significantly improve AF. Typically, this has been accomplished with the use of a pacemaker, where the pacemaker detects the atrial beat, and after a normal delay (0.1-0.2 seconds), triggers a ventricular beat, unless it has already happened—this can be achieved with a single pacing lead with electrodes in the right atrium (to sense) and ventricle (to sense and pace). The “atrial rate” is specific to the rate (measured in beats per unit time) of only the atrial beat.

“Coadministering” or “coadministration” refers to the administration of two or more therapeutic agents together at one time. The two or more therapeutic agents can be coformulated into a single dosage form or “combined dosage unit”, formulated separately and subsequently combined into a combined dosage unit, typically for intravenous administration or oral administration, or the two or more therapeutic agents can be administered separately. The two or more therapeutic agents can also be administered sequentially.

“Intravenous administration” is the administration of substances directly into a vein, or “intravenously”. Compared with other routes of administration, the intravenous (IV) route is the fastest way to deliver fluids and medications throughout the body. An infusion pump can allow precise control over the flow rate and total amount delivered, but in cases where a change in the flow rate would not have serious consequences, or if pumps are not available, the drip is often left to flow simply by placing the bag above the level of the patient and using the clamp to regulate the rate. Alternatively, a rapid infuser can be used if the patient requires a high flow rate and the IV access device is of a large enough diameter to accommodate it. This is either an inflatable cuff placed around the fluid bag to force the fluid into the patient or a similar electrical device that may also heat the fluid being infused. When a patient requires medications only at certain times, intermittent infusion is used, which does not require additional fluid. It can use the same techniques as an intravenous drip (pump or gravity drip), but after the complete dose of medication has been given, the tubing is disconnected from the IV access device. Some medications are also given by IV push or bolus, meaning that a syringe is connected to the IV access device and the medication is injected directly (slowly, if it might irritate the vein or cause a too-rapid effect). Once a medicine has been injected into the fluid stream of the IV tubing there must be some means of ensuring that it gets from the tubing to the patient. Usually this is accomplished by allowing the fluid stream to flow normally and thereby carry the medicine into the bloodstream; however, a second fluid injection is sometimes used, a “flush”, following the injection to push the medicine into the bloodstream more quickly.

“Oral administration” is a route of administration where a substance is taken through the mouth, and includes buccal, sublabial and sublingual administration, as well as enteral administration and that through the respiratory tract, unless made through e.g. tubing so the medication is not in direct contact with any of the oral mucosa. Typical form for the oral administration of therapeutic agents includes the use of tablets or capsules.

A “sustained release formulation” is a formulation which is designed to slowly release a therapeutic agent in the body over an extended period of time, whereas an “immediate release formulation” is a formulation which is designed to quickly release a therapeutic agent in the body over a shortened period of time. In some cases the immediate release formulation may be coated such that the therapeutic agent is only released once it reached the desired target in the body (e.g. the stomach).

“PR interval” is the time interval between the beginning of the P wave and the beginning of the QRS complex of an electrocardiogram that represents the time between the beginning of the contraction of the atria and the beginning of the contraction of the ventricles. The QRS complex is the series of deflections in an electrocardiogram that represent electrical activity generated by ventricular depolarization prior to contraction of the ventricles.

“A-H interval” is the time interval between the onset of the first rapid atrial deflection and the HIS bundle deflection.

The term “prodrug” refers to compounds that include chemical groups which, in vivo, can be converted and/or can be split off from the remainder of the molecule to provide for the active drug, a pharmaceutically acceptable salt thereof, or a biologically active metabolite thereof

### 2. Methods

Generally, the invention relates to a method of treating or preventing atrial fibrillation and/or atrial flutter. The method comprises coadministration of a therapeutically effective amount of ivabradine or a pharmaceutically acceptable salt thereof and a therapeutically effective amount of ranolazine. In one embodiment, either one or both of ranolazine or ivabradine are administered in a synergistically effective amount. The two agents may be administered separately or together in separate or a combined dosage unit. If administered separately, the ranolazine may be administered before or after administration of the ivabradine.

As further discussed in the Examples, evidence of a potent effect of the combination of ranolazine and ivabradine to slow AV conduction and reduce dominant frequency of atrial fibrillation thereby reducing ventricular rate during atrial fibrillation, particularly when the atrial rate is high, as in atrial fibrillation, is shown.

Ranolazine is an anti-ischemic and antianginal agent that has been shown in preclinical and clinical studies to inhibit the late sodium current (I_{Na}) and improve diastolic relaxation. In preclinical studies, ranolazine has also been shown to prevent cellular calcium overload and reduce cardiac electrical and mechanical dysfunction during ischemia. Ranolazine has been shown capable of reducing the dominant frequency of AF (Kumar et al. J Cardiovasc Electrophysiol 2009; 20: 796-802).

Results of several recent studies have demonstrated that ranolazine reduces atrial arrhythmic activity, causes greater inhibition of sodium channels in atrial than in ventricular tissue, prolongs the duration of the action potential (APD90, duration of the action potential at 90% of repolarization) in atria but has minimal or no effect on APD in ventricular myocardium at clinically relevant concentrations (5 and 10 μM), causes significant use-dependent (i.e., the effect of ranolazine was greater at higher rates of pacing) depression of the maximum rate of rise of the action potential upstroke (Vmax) and conduction velocity in atrial myocardium and pulmonary vein sleeves but not in ventricular myocardium (at 5 and 10 μM), and increases the effective refractory period, induced post-repolarization refractoriness, and causes a loss of excitability of the tissue at higher pacing rates in atrial tissue. See Burashnikov et al. Circulation 2007; 116:1449-1457; Song et al. Am J Physiol 2008; 294: H2031-2039; Sicouri et al. Heart Rhythm 2008; 5: 1019-1026; Antzelevitch et al. Circulation 2004; 110: 904-910.

Ivabradine is capable of reducing elevated heart rate during normal, sinus rhythm, in the absence of AF. This effect is achieved through inhibition of the pacemaker current I_{f} at the level of the sinoatrial node (Fox K, et al. Lancet 2008; 372:807-16). However, the view is widespread that ivabradine should have no significant effect on potential non-sinoatrial node pacemaker sites (Du X J, et al. Br J Pharmacol 2004; 142(1):107-12) based on electrophysiological studies by Thollon C, et al. (Eur J Pharmacol (1997 Nov. 19) 339(1):43-51) and Camm A J and Lau C-P (Drugs R&D 2003; 4 (2): 83-89), which showed that ivabradine (0.2 mg/kg, i.v.) lowers heart rate without causing changes in conductivity, refractoriness or repolarization duration of AV node and ventricular Purkinje system. In a review by Savelieva and Camm (Drug Safety 2008; 31(2): 95-107) it was concluded that, “[i]ₙ clinical electrophysiological studies, ivabradine did not affect intra-atrial conduction time or atrial refractoriness” and “[i]vabradine has no blocking effect on the atrioventricular node and is ineffective at reducing heart rate in patients with permanent atrial fibrillation.”

Surprisingly, and as shown in the Examples, the combination of ranolazine and ivabradine synergistically act to slow AV conduction, particularly when the atrial rate is high, as in atrial fibrillation. Moreover, the combination of ranolazine and ivabradine at clinically safe levels decreases ventricular rate during AF by synergistically reducing AV node conduction and AF dominant frequency without QT prolongation or depression in contractility. Targeting these actions offers advantages over conventional nodal agents, which can reduce contractility.

The synergistic effects of the combination of ranolazine and ivabradine were evident in prolongation of PR and A-H intervals. Consequently, ranolazine potentiated ivabradine's reduction in ventricular rate. The drugs did not alter QT or H-V intervals or depress contractility.

The synergistic effects of the combination of ranolazine and ivabradine were also evident in reducing the dominant frequency of atrial fibrillation, a factor in reduction in ventricular rate.

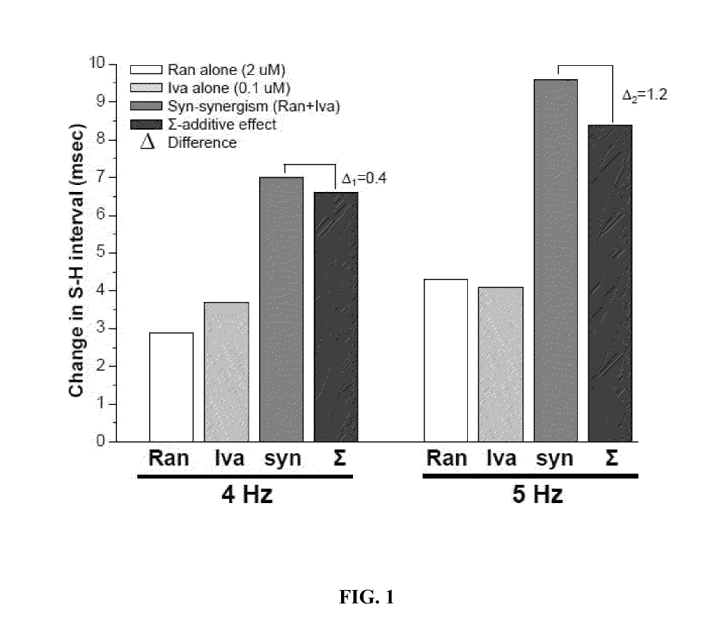

Accordingly, in one embodiment, the disclosure is directed to a method for modulating ventricular rate in a patient in need thereof, said method comprising administering to the patient a therapeutically effective amount of ranolazine and a therapeutically effective amount of ivabradine or a pharmaceutically acceptable salt thereof. In one embodiment, the AV conduction is slowed when atrial rate is high, i.e., greater than 100 beats per minute). It is contemplated that this may be beneficial to provide control of the ventricular rate during atrial fibrillation (see Example 1, FIG. 1). This confirms the effect of the drug combination to provide control of the ventricular rate when the atrial rate is increased, such as during AF (see Example 1, FIG. 1).

In still another embodiment, provided is a method for providing rate control of the ventricles in a patient in need thereof, said method comprising administering to the patient a therapeutically effective amount of ranolazine and a therapeutically effective amount of ivabradine or a pharmaceutically acceptable salt thereof.

In one embodiment, provided is a method for maintaining or improving cardiac function during atrial fibrillation in a human patient in need thereof, comprising administering to the patient a therapeutically effective amount of ivabradine or a pharmaceutically acceptable salt thereof and a therapeutically effective amount of ranolazine. In some embodiments, the improvement is an improvement in left ventricular function. The improvement in left ventricular function can be observed by several known methods, such as echocardiographically, or as an improvement in arterial blood pressure. As described in Example 2, the combined administration of ivabradine and ranolazine maintains or even improves the arterial blood pressure.

Therefore, in a further embodiment, provided is a method for maintaining or improving cardiac mechanical function and arterial blood pressure during atrial fibrillation in a human patient in need thereof, comprising administering to the patient a therapeutically effective amount of ivabradine or a pharmaceutically acceptable salt thereof and a therapeutically effective amount of ranolazine. It is contemplated that the improvement in arterial blood pressure is a direct consequence of enhanced left ventricular function. By alleviating low arterial blood pressure (hypotension), the impaired perfusion of blood to the brain and vital organs can be mitigated. This is a significant observation that distinguishes the presently disclosed methods from other, currently used therapies. For example, other antiarrhythmic drugs, such as diltiazem, decrease arterial blood pressure during therapy with attendant risks.

In one embodiment, the disclosure is directed to a method for increasing the A-H interval in a human patient in need thereof, comprising administering to the patient a therapeutically effective amount of ivabradine and a therapeutically effective amount of the ranolazine. A-H interval is a surrogate marker for AV nodal conduction. The longer the A-H interval, the more time is required for an impulse to be conducted through the AV node, thereby slowing conduction. By prolonging the A-H (and/or S-H) interval, the combination effectively slows AV nodal conduction and reduces ventricular rate.

In another embodiment, the disclosure is directed to a method for increasing the PR interval in a human patient in need thereof, comprising administering to the patient a therapeutically effective amount of ivabradine and a therapeutically effective amount of the ranolazine. PR interval is a surrogate marker for AV nodal conduction. The longer the PR interval, the more time is required for an impulse to be conducted through the AV node, thereby slowing conduction. By prolonging the PR interval, the combination effectively slows AV nodal conduction and reduces ventricular rate.

In another embodiment, the disclosure is directed to a method of decreasing dominant frequency of AF in a patient in need thereof, said method comprising administering to the patient a therapeutically effective amount of ranolazine and a therapeutically effective amount of ivabradine or a pharmaceutically acceptable salt thereof.

In another embodiment, the disclosure is directed to a method for:

i) controlling ventricular rate during atrial fibrillation or atrial flutter;

ii) improving left ventricular function during atrial fibrillation or atrial flutter;

iii) decreasing dominant frequency of atrial fibrillation;

iv) increasing the A-H interval;

v) increasing the PR interval;

vi) modulating ventricular rate;

vii) modulating atrial rate;

viii) providing rate control of the ventricles and/or atria;

ix) maintaining or improving cardiac function during atrial fibrillation;

x) maintaining or improving arterial blood pressure during atrial fibrillation;

xi) modulating electrical and structural remodeling;

xii) treating or preventing atrial fibrillation; or

xiii) treating or preventing atrial flutter,

in a human patient, wherein the method comprises administering to the human patient a therapeutically effective amount of ivabradine or a pharmaceutically acceptable salt thereof and a therapeutically effective amount of ranolazine.

Another measurement which is an indicator of AV nodal conduction is the S-H interval. In one embodiment, the disclosure is directed to a method for increasing the S-H interval in a human patient in need thereof, comprising administering to the patient a therapeutically effective amount of ivabradine or a pharmaceutically acceptable salt thereof and a therapeutically effective amount of the ranolazine. In one embodiment, the S-H interval is increased when the atrial rate is high.

In another embodiment, the disclosure is directed to a use of a therapeutically effective amount of ivabradine or a pharmaceutically acceptable salt thereof and a therapeutically effective amount of ranolazine in the preparation of a medicament for treating or preventing atrial fibrillation.

In another embodiment, the disclosure is directed to a therapeutically effective amount of ivabradine or a pharmaceutically acceptable salt thereof and a therapeutically effective amount of ranolazine for use in therapy.

As mentioned above, prolonged atrial fibrillation often results in development of congestive heart failure and/or stroke. In addition, patients with atrial fibrillation have increased risks of hospitalization and death. Thus, as a consequence of treating and preventing atrial fibrillation, the combination therapy is expected to reduce hospitalization, the development of heart failure, and incidence of stroke. It is further contemplated that by reducing or preventing atrial fibrillation, emboli and blood clot formation is attenuated or reduced. Accordingly, in one aspect, the disclosure is directed to the method of preventing congestive heart failure and/or stroke in a patient by coadministration of ivabradine or a salt thereof and ranolazine.

In embodiments of the above methods, the patient in need thereof is one that suffers from atrial fibrillation. In further embodiments of the above-disclosed methods, the arterial blood pressure is maintained or modulated in the human patient. In a related embodiment, the arterial blood pressure is increased.

In embodiments of the above methods, it is contemplated that a derivative or a prodrug of ivabradine may be used in combination with ranolazine.

### 2.1 Dosing

For all of the methods just described, it is contemplated that at least one of either ranolazine or ivabradine or pharmaceutically acceptable salt thereof is administered in a therapeutically effective amount. In some embodiments, the ivabradine is administered in a synergistically effective dose and ranolazine is administered in a therapeutically effective dose. In other embodiments, ranolazine is administered in a synergistically effective dose and ivabradine is administered in a therapeutically effective dose. In still other embodiments, both ranolazine and ivabradine are administered in a synergistically effective dose.

In some embodiments, ivabradine or a pharmaceutically acceptable salt thereof and ranolazine are administered separately.

Ranolazine and ivabradine may be given to the patient in either single or multiple doses by any of the accepted modes of administration of agents having similar utilities, for example as described in those patents and patent applications incorporated by reference, including buccal, by intra-arterial injection, intravenously, intraperitoneally, parenterally, intramuscularly, subcutaneously, orally, or via an impregnated or coated device such as a stent, for example, or an artery-inserted cylindrical polymer. In one embodiment, ranolazine and ivabradine or a pharmaceutically acceptable salt thereof are administered intravenously.

In one embodiment, ranolazine and ivabradine or a pharmaceutically acceptable salt thereof are administered orally. Ivabradine or a pharmaceutically acceptable salt thereof and ranolazine may also be administered as a combined dosage unit, such as, for example, in a tablet.

As mentioned above, ivabradine or a pharmaceutically acceptable salt thereof and ranolazine may be administered in a therapeutically effective amount or a synergistically effective amount. Therefore, in some embodiments, the amount of ranolazine administered is from about 50 mg to about 3000 mg daily or from about 50 mg to about 2500 mg daily, or from about 50 mg to about 2000 mg daily, or from about 50 mg to about 1500 mg daily, or from about 50 mg to about 1000 mg daily, or from about 50 mg to about 500 mg daily, or from about 50 mg to about 200 mg daily. Further, the amount of ivabradine or pharmaceutically acceptable salt thereof administered is from about 1 mg to about 50 mg daily, or from about 1 mg to about 40 mg daily, or about 1 mg to about 30 mg daily, or from about 1 mg to about 20 mg daily, or from about 1 mg to about 10 mg daily. These aggregate daily doses may be administered to the patient either once or twice a day.

As mentioned above, ivabradine or a pharmaceutically acceptable salt thereof and ranolazine may be administered intravenously. The amount of ranolazine administered is from about 10 mg/hr to about 200 mg/hr, or from about 10 mg/hr to about 100 mg/hr or from about 10 mg/hr to about 50 mg/hr. The amount of ivabradine is from about 0.01 mg/kg to about 1 mg/kg, from about 0.01 mg/kg to about 0.25 mg/kg, or from about 0.01 mg/kg to about 0.1 mg/kg. In some embodiments, the ivabradine or a pharmaceutically acceptable salt thereof is administered as a bolus injection.

Additionally, it is contemplated that ranolazine is administered as a sustained release formulation and/or ivabradine or pharmaceutically acceptable salt thereof is administered as an immediate release or sustained release formulation. This is more thoroughly discussed in the next section.

### 3. Active Ingredients and Compositions

### 3.1 Ranolazine

U.S. Pat. No. 4,567,264, discloses ranolazine, (±)-N-(2,6-dimethylphenyl)-4-[2-hydroxy-3-(2-methoxyphenoxy)-propyl]-1-piperazineacetamide, and pharmaceutically acceptable salts thereof, and their use in the treatment of cardiovascular diseases, including arrhythmias, variant and exercise-induced angina, and myocardial infarction.

This patent also discloses intravenous (IV) formulations of ranolazine dihydrochloride further comprising propylene glycol, polyethylene glycol 400, Tween 80 and 0.9% saline.

U.S. Pat. No. 5,506,229, discloses the use of ranolazine and its pharmaceutically acceptable salts and esters for the treatment of tissues experiencing a physical or chemical insult, including cardioplegia, hypoxic or reperfusion injury to cardiac or skeletal muscle or brain tissue, and for use in transplants. Oral and parenteral formulations are disclosed, including controlled release formulations. In particular, Example 7D of U.S. Pat. No. 5,506,229 describes a controlled release formulation in capsule form comprising microspheres of ranolazine and microcrystalline cellulose coated with release controlling polymers. This patent also discloses IV ranolazine formulations which at the low end comprise 5 mg ranolazine per milliliter of an IV solution containing about 5% by weight dextrose. And at the high end, there is disclosed an IV solution containing 200 mg ranolazine per milliliter of an IV solution containing about 4% by weight dextrose.

The presently preferred route of administration for ranolazine is oral. A typical oral dosage form is a compressed tablet, a hard gelatin capsule filled with a powder mix or granulate, or a soft gelatin capsule (softgel) filled with a solution or suspension. U.S. Pat. No. 5,472,707, discloses a high-dose oral formulation employing supercooled liquid ranolazine as a fill solution for a hard gelatin capsule or softgel.

U.S. Pat. No. 6,503,911, discloses sustained release formulations that overcome the problem of affording a satisfactory plasma level of ranolazine while the formulation travels through both an acidic environment in the stomach and a more basic environment through the intestine, and has proven to be very effective in providing the plasma levels that are necessary for the treatment of angina and other cardiovascular diseases.

U.S. Pat. No. 6,852,724, discloses methods of treating cardiovascular diseases, including arrhythmias variant and exercise-induced angina and myocardial infarction.

U.S. Patent Application Publication Number 2006/0177502, discloses oral sustained release dosage forms in which the ranolazine is present in 35-50%, preferably 40-45% ranolazine. In one embodiment the ranolazine sustained release formulations of the disclosure include a pH dependent binder; a pH independent binder; and one or more pharmaceutically acceptable excipients. Suitable pH dependent binders include, but are not limited to, a methacrylic acid copolymer, for example Eudragit® (Eudragit® L100-55, pseudolatex of Eudragit® L100-55, and the like) partially neutralized with a strong base, for example, sodium hydroxide, potassium hydroxide, or ammonium hydroxide, in a quantity sufficient to neutralize the methacrylic acid copolymer to an extent of about 1-20%, for example about 3-6%. Suitable pH independent binders include, but are not limited to, hydroxypropylmethylcellulose (HPMC), for example Methocel® E10M Premium CR grade HPMC or Methocel® E4M Premium HPMC. Suitable pharmaceutically acceptable excipients include magnesium stearate and microcrystalline cellulose (Avicel® pH101).

In one embodiment, the methods of the disclosure employ a pharmaceutically acceptable salt of ranolazine.

### 3.2 Ivabradine

U.S. Pat. No. 5,296,482 discloses ivabradine, 3-{3-[{[(7S)-3,4-dimethoxybicyclo[4.2.0]octa-1,3,5-trien-7-yl]methyl}-(methyl)amino]propyl}-7,8-dimethoxy-1,3,4,5-tetrahydro-2H-3-benzapenin-2-one, the preparation and therapeutic use of ivabradine and pharmaceutically acceptable salts thereof, such as ivabradine hydrochloride.

Ivabradine hydrochloride is an example of a commonly used pharmaceutically acceptable salt of ivabradine. It is contemplated that a derivative or a prodrug of ivabradine may be used in the methods of the disclosure.

In one embodiment, the methods of the disclosure employ a tablet comprising ivabradine. The tablet optionally additionally comprises one or more pharmaceutical excipients. The tablet may also optionally comprise ranolazine.

### 3.3 Pharmaceutical Formulations

As mentioned above, ivabradine and ranolazine may be coadministered, meaning that the two active ingredients may be formulated separately but administered at similar times (i.e., either together or one after the other). Coadministered also means that ivabradine and ranolazine may be coformulated into a combined dosage unit. Accordingly, in one embodiment, the disclosure is directed to pharmaceutical formulations comprising a therapeutically effective amount of ivabradine or pharmaceutically acceptable salt thereof, a therapeutically effective amount of ranolazine, and a pharmaceutically acceptable carrier.

In another embodiment, the formulation comprises a synergistically effective amount of ranolazine and/or ivabradine or pharmaceutically acceptable salt thereof. In certain embodiments, the formulations are formulated for either intravenous or oral administration. In still other embodiment, the two active ingredients are coformulated into a combined dosage unit. In still yet other embodiments, the two active ingredients are formulated separately for coadministration.

### 3.4 Coformulations

In certain embodiments of the present disclosure, the ranolazine and ivabradine are coformulated into a combined dosage unit or unitary dosage form suitable for oral administration. In certain embodiments, the ranolazine is formulated as a sustained release formulation. In certain embodiments, the ivabradine is formulated for immediate release or sustained release.

In one such embodiment, a pharmaceutically acceptable composition comprising ranolazine is placed in a portion of the tablet which is separate from, but in contact with, the portion of the tablet containing a pharmaceutically acceptable composition comprising ivabradine. It will be understood that the unitary dosage form may comprise simply compressing the ranolazine composition and the ivabradine composition into a multilayer tablet or conventionally processed into other conventional unitary dosage forms such as a capsules. The multilayer tablets and capsules suitable for use in the present disclosure can be fabricated using methods known in the art using standard machinery.

The tablets may comprise two layers, i.e. a first layer which comprises the ivabradine and is formulated for immediate release or sustained release, and a second layer which comprises the ranolazine and is formulated for sustained release. Alternatively, the multilayer tablet may comprise an inner layer and an outer layer, where the inner layer comprises the sustained release ranolazine formulation and where the outer layer comprises the immediate release or sustained release ivabradine layer. In another embodiment, the ranolazine and ivabradine are coformulated into a capsule, where the capsule allows for the immediate release or sustained release of ivabradine and the sustained release of ranolazine. For example, the capsule may contain granules of both ivabradine and ranolazine, where the granules have been formulated such that the ivabradine is available for immediate release or sustained release and the ranolazine is formulated for sustained release. Alternatively, the capsule may contain a liquid immediate release or sustained release formulation of ivabradine and a solid sustained release formulation of ranolazine. However, such embodiments are exemplary and are not intended to limit the formulations of the present disclosure.

A multilayer tablet can be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active agent or dispersing agent. Molded tablets may be made by molding in a suitable machine a mixture of the powdered active ingredient moistened with an inert liquid diluent. The tablets may optionally be coated or scored.

The tablets may contain one or more agents including sweetening agents, flavoring agents, coloring agents and preserving agents, in order to provide a palatable preparation. Tablets containing the active ingredients in admixture with non-toxic pharmaceutically acceptable excipients which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

### 3.5 Additional Formulations

Formulations contemplated by the present disclosure may also be for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present disclosure. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. The same formulations are contemplated for separate administration of ranolazine and ivabradine.

Sterile injectable solutions are prepared by incorporating the component in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The ideal forms of the apparatus for administration of the novel combinations for atrial fibrillation and other methods of the disclosure consist therefore of (1) either a syringe comprising 2 compartments containing the 2 active substances ready for use or (2) a kit containing two syringes ready for use.

In making pharmaceutical compositions that include ranolazine and ivabradine, the active ingredients are usually diluted by an excipient or carrier and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compounds, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the disclosure can be formulated so as to provide quick, sustained or delayed release of the active ingredient after administration to the patient by employing procedures known in the art. As discussed above, given the reduced bioavailabity of ranolazine, sustained release formulations are generally preferred. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345.

The compositions are preferably formulated in a unit dosage form. The term “unit dosage forms” or “combined dosage unit” refers to physically discrete units suitable as unitary dosages for human subjects and other mammals, each unit containing a predetermined quantity of the active materials calculated to produce the desired therapeutic effect, in association with a suitable pharmaceutical excipient (e.g., a tablet, capsule, ampoule). The active agents of the disclosure are effective over a wide dosage range and are generally administered in a pharmaceutically effective amount. It will be understood, however, that the amount of each active agent actually administered will be determined by a physician, in the light of the relevant circumstances, including the condition to be treated, the chosen route of administration, the actual compounds administered and their relative activity, the age, weight, and response of the individual patient, the severity of the patient's symptoms, and the like.

For preparing solid compositions such as tablets, the principal active ingredients are mixed with a pharmaceutical excipient to form a solid preformulation composition containing a homogeneous mixture of a compound of the present disclosure. When referring to these preformulation compositions as homogeneous, it is meant that the active ingredients are dispersed evenly throughout the composition so that the composition may be readily subdivided into equally effective unit dosage forms such as tablets, pills and capsules.

The tablets or pills of the present disclosure may be coated or otherwise compounded to provide a dosage form affording the advantage of prolonged action, or to protect from the acid conditions of the stomach. For example, the tablet or pill can comprise an inner dosage and an outer dosage element, the latter being in the form of an envelope over the former. Ranolazine and the co-administered agent(s) can be separated by an enteric layer that serves to resist disintegration in the stomach and permit the inner element to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Additional embodiments of the disclosure include kits comprising a therapeutically effective amount of ranolazine and a therapeutically effective amount of ivabradine or a pharmaceutically acceptable salt thereof.

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### EXAMPLES

Ivabradine as used in this disclosure is well known in the art, and is commercially available. Ranolazine is also commercially available or may be prepared by conventional methods such as in the manner disclosed in U.S. Pat. No. 4,567,264, the entire disclosure of which is hereby incorporated by reference. Additionally, the abbreviations used throughout have the following meanings:
g=gram
mM=millimolar
° C.=degrees centigrade
min=minute
CPP=coronary perfusion pressure
S-H=stimulus to His
Hz=hertz
Iva=ivabradine
Ran=Ranolazine
μM=micromolar
kg=kilogram
SEM=standard error of the mean
h=hour
i.v.=intravenous
ACh=acetylcholine
AF=atrial fibrillation
RA=right atrium
Msec or ms=millisecond
A-H=atrium to His
bpm=beats per minute
MAP=mean arterial pressure
NS=not significant
BL=baseline
mV=millivolt
mL=milliliter
nM=nanomolar
nG=nanogram
dP/dt=rate of pressure change over time in the ventricle

### Example 1

Synergistic Effects of Ivabradine and Ranolazine on the S-H Interval in Guinea Pig Isolated Hearts

To test the combined effects of ranolazine and ivabradine on AV conduction, the following experimental procedures were employed.

### Experimental Procedures

### 1. Guinea Pig Heart Isolation and Perfusion

Guinea pigs (Hartley) of either sex weighing 300-350 g were anesthetized by inhalation of isoflurane. The chest of a guinea pig was cut open, and the heart was quickly removed and rinsed in ice-cold modified Krebs-Henseleit (K-H) solution. The contents of the modified K-H solution were (in mM) 117.9 NaCl, 4.8 KCl, 2.5 CaCl₂, 1.18 MgSO₄, 1.2 KH₂PO₄, 0.5 Na₂ EDTA, 0.14 ascorbic acid, 5.5 dextrose, 2.0 pyruvic acid (sodium salt), and 25 NaHCO₃. The K-H solution was continuously gassed with 95% O₂-5% CO₂, and the pH was adjusted to a value of 7.4.

To perfuse the heart by the Langendorff method, the transected aorta was slid onto a glass cannula and secured by a ligature. Retrograde perfusion of the aorta was initiated immediately at a constant flow of 10 mL/min with modified K-H solution warmed to 37.0±0.5° C. A side port in the cannula was used to connect the perfusion line to a pressure transducer (AD Instruments, Australia) for measurement of coronary perfusion pressure (CPP). To facilitate the exit of fluid from the left ventricle, the leaflets of the mitral valve were trimmed with fine spring-handled scissors. Hearts were allowed to beat spontaneously in experiments to measure heart rate, or paced at a constant rate using external electrodes, in experiments to measure AV conduction time. After completion of dissection and instrumentation, stimulus-to-H is bundle (S-H) interval was monitored continuously. Each heart was allowed to equilibrate for 20-40 min before the administration of drug. Experimental interventions were always preceded and followed by control measurements.

### 2. Measurement of S-H Interval

To facilitate the recording of a drug effect on the S-H interval, parts of the left and right atrial tissues, including the region of the sinoatrial node, were removed, both to decrease the spontaneous heart rate and to expose the atrial septum for electrode placement. A bipolar Teflon-coated electrode was placed in the wall of the intra-atrial septum to pace the heart. Hearts were electrically paced at a fixed rate of 3.2 Hz. Stimuli were provided by a stimulation generator (model 48, Grass Instruments, W. Warwick, R I) and delivered to the heart through a stimulus isolation unit as square wave pulses of 3 msec duration and at least twice the threshold intensity.

A His bundle electrogram was recorded using a Teflon-coated unipolar electrode placed in the right side of the interatrial septum adjacent to the AV junction. The signal was displayed continuously in real time on an oscilloscope screen (Tektronix Inc., Beaverton, Oreg.) at a sweep speed of 10 msec/cm and on a computer monitor. The duration of time from the first pacing artifact to the maximum upward deflection of the His bundle signal was used as the S-H interval.

### 3. Experimental Protocol for Isolated, Perfused Heart Experiments

At the beginning of an experiment, a heart was perfused with saline until either the heart rate or the S-H interval remained constant for at least 5-10 minutes.

Ivabradine (Iva), ranolazine (Ran) or the combination thereof was infused to the hearts. Each concentration of Iva was infused for about 20 min to allow a steady-state response to be recorded, whereas each concentration of ranolazine was infused for 10 min to allow a steady-state response to be recorded. Then drug administration was discontinued and saline administration was initiated to begin drug washout.

### Results

### Effects of Ivabradine, Ranolazine and the Combination on AV Nodal Conduction (S-H Interval) in Guinea Pig Hearts

Ranolazine is a weak antagonist of beta-adrenergic receptors (activation of which can increase AV conduction) and a weak voltage- and rate-dependent sodium channel blocker, but has not been shown to alter AV nodal conduction. Ivabradine is reported to inhibit the I_{f}-ion current, which, as an intrinsic pacemaker in the heart, controls the spontaneous diastolic depolarization in the sinoatrial node and thus regulates the heart rate. To determine the effects of both drugs alone and in combination, the duration of the S-H interval (a surrogate for the velocity of electrical impulse conduction through the AV node) was measured in the absence and presence of drug(s). Either ivabradine or ranolazine caused a small slowing of AV conduction without causing second-degree AV block (i.e., dropped beats). As shown in FIG. 1, ivabradine (0.1 μM) or ranolazine (2 μM) caused a small but significant increase in the S-H interval compared to control (no drug) at pacing rate of 4 and 5 Hz (n=2 for Ran and Iva alone, FIG. 1). The greatest effect of the drug combination was observed at the highest pacing rate (i.e., 5 Hz). A combination of ivabradine and ranolazine caused a much greater increase in the S-H interval (n=5, FIG. 1). This increase of the S-H interval caused by the combination of ranolazine and ivabradine was greater than the calculated sum of the individual effects of both drugs (i.e., Σ(R+I), FIG. 1). The results suggest that the combination of ranolazine and ivabradine may have a greater effect to slow AV conduction when the atrial rate is high, as in atrial fibrillation. This action may be beneficial to provide control of the ventricular rate during atrial fibrillation.

### Example 2

### Synergistic Effects of Ivabradine and Ranolazine on the PR and A-H Intervals, Dominant Frequency and Arterial Blood Pressure in an Acetylcholine

### Mediated AF Model in Pigs

To test the combined effects of ranolazine and ivabradine on ventricular rate and AV node conduction, the following experimental procedures were employed.

### Experimental Procedures

### 1. Yorkshire Pig Experimental Procedures

Data were gathered from male Yorkshire pigs (n=16) weighing 35±2 kg (mean±SEM). The animals were preanesthetized with telazol (4.7 mg/kg, intramuscularly) and then anesthetized with alpha-chloralose (100 mg/kg, i.v., bolus followed by 40 mg/kg/h, i.v., continuous infusion). The animals were intubated and ventilation was maintained between 10-16 breaths/min with volumes between 400-500 mL. Vital signs including core body temperature, heart rate, and oxygen saturation were continuously monitored.

The right and left femoral veins were cannulated with 7Fr introducer sheaths using the Seldinger technique to insert catheters in the right atrial (RA) appendage for pacing and for positioning the intrapericardial catheter. Arterial blood pressure was continuously monitored from a femoral arterial sheath, and intravenous fluids were administered through the ear vein. The electrocardiogram (ECG) was recorded with a Prucka Cardiolab workstation (GE Medical Systems, Milwaukee, Wis.) from atrial and ventricular sites.

Ivabradine was administered at a dose of 0.25 mg/kg, i.v., infused over 5 minutes via a 7Fr sheath inserted into the right femoral vein and at a 0.1 mg/kg dose, i.v., dose which has recently been shown to exert use-dependent slowing of AV node conduction and to reduce ventricular rate during AF (Verrier et al. JACC 2014; 63:A377 2014) to determine whether a synergistic effect was demonstrable at this lower dose. Ranolazine was given as a 2.4 mg/kg, i.v., bolus followed by continuous infusion of 0.135 mg/kg/min, i.v.

### 2. Cardiac Catheterization

A nonsteerable, quadripolar electrode catheter (Bard Electrophysiology, Lowell Mass.) was placed in the right atrium (RA) via the femoral vein and a second quadripolar electrode catheter in the right ventricle via the jugular vein. A bipolar electrode was placed in the non-coronary cusp of the aorta through the left carotid artery to record the His bundle electrogram. A pigtail catheter was inserted in the left ventricle via the left femoral artery to record and calculate contractility [left ventricular dP/dt]. Transatrial access was employed to deliver acetylcholine (ACh) into the pericardial space. Specifically, a small puncture was made in the RA appendage with the stiff end of a coronary angioplasty guidewire (0.014-inch Wizdom guidewire, Cordis, Miami, Fla., USA) placed within the lumen of a soft infusion catheter (0.038-inch SOS straight-tip, open-ended angiographic guidewire, Bard Electrophysiology). This wire-within-wire system was advanced as a unit into an 8Fr multipurpose guide catheter (MP2, Boston Scientific, Boston Mass.) previously positioned in the RA appendage via a femoral vein under fluoroscopic guidance and into the pericardial space. The infusion catheter was left in the pericardial space for delivery of ACh and the inner guidewire was removed. Conformation of the infusion catheter on fluoroscopy to the curvature of the heart verified its location within the pericardial space. All of the pericardial fluid was then aspirated with the infusion catheter. Intrapericardial access is associated with a hematocrit <2%, indicating minimum trauma from the atrial puncture (Kumar et al., J Cardiovasc Electrophysiol 2009; 20:796-802). The presence of acetylcholinesterase can rapidly degrade ACh and prevent a sustained response to this neurotransmitter.

### 3. Atrial Fibrillation Induction and Analysis

Atrial fibrillation (AF) was defined as an irregular atrial rhythm with an average cycle length <150 msec at all atrial sites, whereas atrial flutter was defined as a regular atrial rhythm with fixed cycle length >150 msec at all atrial sites. After baseline electrical testing was performed, AF initiation was attempted by decremental burst pacing at each atrial site down to loss of 1:1 capture. ACh (1 mL of 100 mM solution) along with a 1-mL saline flush was injected into the pericardial space via the transatrial infusion catheter. After ACh injection, AF was reliably induced with burst atrial pacing. After 5 minutes of AF, the pericardial space was lavaged with saline (20 mL) via the transatrial pericardial catheter. At the end of lavage, no further pericardial fluid could be removed. Lavage was performed after 5 minutes of AF in order to minimize the mild systemic hypotension that was occasionally associated with ACh administration. The time to spontaneous termination of AF was recorded.

### 4. Dominant Frequency

Dominant frequency of AF was analyzed during the 6th minute after AF initiation using the intracardiac electrograms from the right atrial appendage. The data files were downloaded from the Cardiolab workstation at 977 samples/sec and imported into MATLAB (The MathWorks, Inc., Natick Mass.). Dominant frequency was determined by Fast Fourier Transform analysis of 4,096 spectra from 4-second segments of ECG data and was defined as the frequency with the highest power (Everett et al. IEEE Trans Biomed Eng 2001; 48:969-978).

### 5. Study Protocol

The electrical stimulation protocol described below was performed in the control state and after the ivabradine bolus and after ranolazine administration and the combination of ranolazine and ivabradine. Burst pacing was performed after ACh injection into the pericardium and AF was invariably elicited. Ventricular rate during AF was determined by counting the R waves during the 6th minute of AF. Two episodes of AF separated by 10 min were induced to establish control levels.

### 6. Electrical Testing

A Bloom stimulator (Bloom Associates, Reading Pa.) was used to deliver constant-current rectangular stimuli for fixed-rate pacing as well as for delivering premature stimuli. To elicit AF, a burst pacing stimulation with a cycle length of 200 msec was performed. A-H, His-ventricular (H-V), PR and QT interval measurements were made during right atrial pacing at 130 and 180 beats/min.

### 7. Statistics

The statistical tests were carried out with a SAS statistical package (SAS Institute, Cary, N.C.). Effects on electrophysiologic properties were determined by repeated measures analysis of variance (ANOVA) followed by post-hoc t-tests with Bonferroni correction for multiple comparisons. For mean arterial pressure, heart rate, and dominant frequency during atrial fibrillation, we used paired t-tests to compare effects of agents to baseline. All data are reported as mean±SEM, with P<0.05 considered significant.

### 8. Plasma Level Determination

Blood samples were collected in sodium heparin tubes at 0 and 30 minutes following ranolazine bolus administration and at 0, 15, and 30 minutes after ivabradine bolus administration. The samples were centrifuged and frozen at −80° C. until drug level determination was performed. Concentrations of ivabradine and ranolazine in plasma were determined at Gilead Sciences, Inc. (Foster City Calif.). Plasma was analyzed using a high-performance liquid chromatography-tandem mass spectrometric assay (LC/MS/MS). The quantification limit of ivabradine was 4 nM (1.87 ng/mL). The dynamic range of quantification was 4-10 000 nM (1.87-4686 ng/mL). Ranolazine and an internal standard were extracted with the protein precipitation extraction method. The dynamic range of quantification for ranolazine was 45.8-23,400.

### Results

### 1. Effects of Ranolazine and the Combination of Ivabradine and Ranolazine on AV Nodal Conduction (A-H Interval) in Acetylcholine

### Mediated AF in Pigs

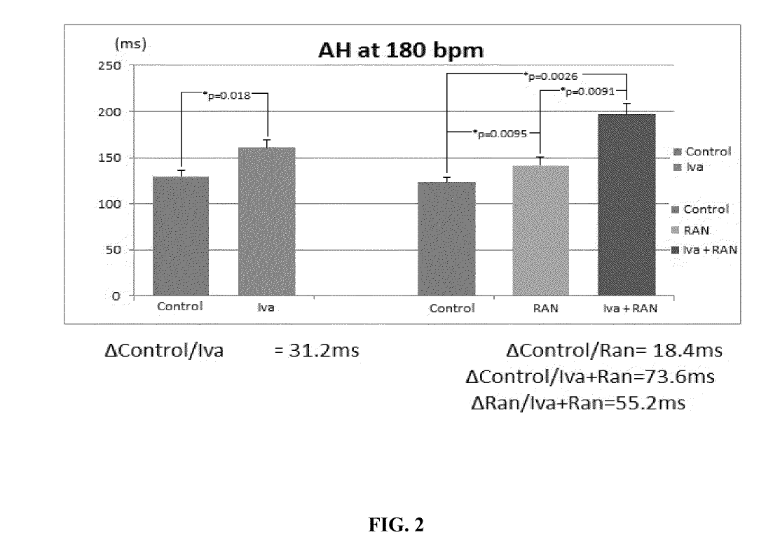

The combination of ranolazine and ivabradine increases the A-H interval at a pacing rate of 180 beat/min (FIG. 2). This interval represents the conduction time between the atrium and AV node as documented by signals from the atrium and His bundle electrograms. The effect is greater when the rate is increased. FIG. 2 demonstrates that the effects on A-H interval are more than additive. Specifically, ivabradine increases A-H interval by 31 msec. Ranolazine increases A-H interval by 18 msec. Thus, their individual additive effects are 49 msec. The effect of the agents in combination is 74 msec, which is greater than the contributions of the drugs given separately. These findings suggest that the combination has synergistic effects by slowing AV node conduction and thereby reducing the ventricular rate during a relatively high atrial pacing rate (180 bpm), as in atrial fibrillation.

In larger series of experiments, it was found that, ivabradine (0.25 mg/kg) increases A-H interval by 22.0±3.5 msec (p=0.007). Ranolazine increase A-H interval by 13.8±3.9 msec but the effect was not significant (p=0.082). Thus, their additive effects total 35.8 msec. The effect of the agents in combination is 50.5±6.2 msec (p=0.0022), which is unexpectedly greater than the contributions of the drugs given separately. These findings confirm that the combination has a greater effect in increasing A-H interval during a relatively high atrial pacing rate (180 bpm), as in atrial fibrillation. Similar synergistic effects on A-H interval are produced during atrial pacing at 130 beats/min and by administration of a lower 0.1-mg/kg dose of ivabradine in combination with ranolazine.

### 2. Effects of Ranolazine and the Combination of Ivabradine and Ranolazine on Mean Arterial Blood Pressure During Acetylcholine

### Mediated AF in Pigs

During atrial fibrillation, wherein electrical activity is extremely rapid, in the range of 350-500 bpm, the AV node, which is the way station for activation of the ventricles, the main chambers of the heart, is literally bombarded by electrical stimuli from the atria, resulting in inefficient pumping action of the ventricles, thereby leading to low arterial blood pressure. This is an unhealthy state for the patient, as there is poor perfusion of the brain and other vital organs including the heart, kidneys and gut. In some cases the consequences can be dire, as stroke, heart attack, and damage to other vital organs may occur. Thus, a strategic target for improving the pumping action of the heart is to enhance the electrical filtering capacity of the AV node.

Contemporary drugs, referred to as “AV nodal agents,” such as the calcium channel blocking agent diltiazem or the beta-adrenergic receptor blocking agent metoprolol, slow conduction through the AV node but at the same time depress the strength of contraction of the ventricles as their actions are not selective for the AV node.

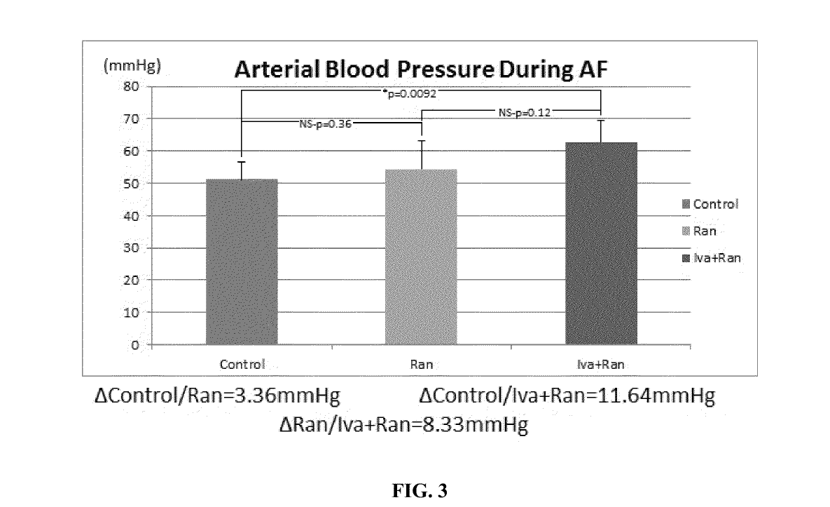

The combination of ranolazine and ivabradine (but not single drug administration) shows a significant improvement in mean arterial blood pressure maintenance during AF (FIG. 3). These results demonstrate that the RAN/IVA combination therapy is a means to enhance electrical filtering capacity of the AV node without adversely affecting the mechanical function of the heart. Thus, an essential observation is the finding that ranolazine and ivabradine exert a synergistic effect of slowing conduction through the AV node without depressing—and in fact while improving—arterial blood pressure during AF.

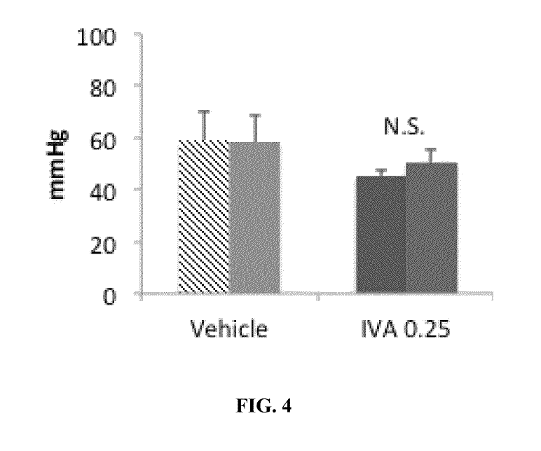

Synergy is also evident in the important aspect of arterial blood pressure maintenance during AF. Neither ivabradine (FIG. 4) nor ranolazine (FIG. 3) given alone improves arterial blood pressure during AF. However, the combination exerts a highly significant improvement (FIG. 3). It is believed that the improvement in arterial blood pressure is a direct consequence of enhanced left ventricular function. The ventricles beat erratically and ineffectively during AF (data not shown). With ranolazine alone, there is some improvement. Following administration of both ranolazine and ivabradine, the pumping action is strong and efficient. The more regular beating and resulting improved mechanical function account for the improvement in arterial blood pressure. By alleviating low arterial blood pressure (hypotension), the impaired perfusion to the brain and vital organs can be mitigated.

In summary, these results demonstrate that combined administration of ranolazine and ivabradine provides a novel therapeutic strategy for reducing ventricular rate during atrial fibrillation without depressing contractility. This effect is associated with the major benefit of improving arterial blood pressure and the mechanical function of the heart during AF. These findings have major implications as they can improve health and decrease morbidity and mortality in patients who are afflicted by AF.

### 3. Effects of Ivabradine, Ranolazine and the Combination of Ivabradine and Ranolazine on Hemodynamic Changes

At the time of electrical testing, ivabradine (0.25 mg/kg) alone lowered sinus rate from 111±4.0 to 90±3.3 beats/min (p=0.003). Ranolazine alone did not affect sinus rate (from 105±5.7 to 96±3.9 beats/min, NS) but with the addition of ivabradine, sinus rate decreased by 32 beats/min (to 73±2.9 beats/min, p=0.002), a change that was greater than the additive effects of the agents alone. Neither ivabradine nor ranolazine given separately altered mean arterial pressure during sinus rhythm. Left ventricular (LV) dP/dt was not altered by either drug or their combination (from baseline 2156±647.4 to 2342±486.5 mmHg/sec at 30 min after ivabradine alone; from baseline 1864±109.1 to 1594±429.0 mmHg/sec at 30 min after ranolazine alone and to 1890±233.4 mmHg/sec at 30 min after the addition of ivabradine during pacing at 180 beats/min, NS for all).

### 4. Effects of Ivabradine, Ranolazine and the Combination of Ivabradine and Ranolazine on PR and QT Intervals

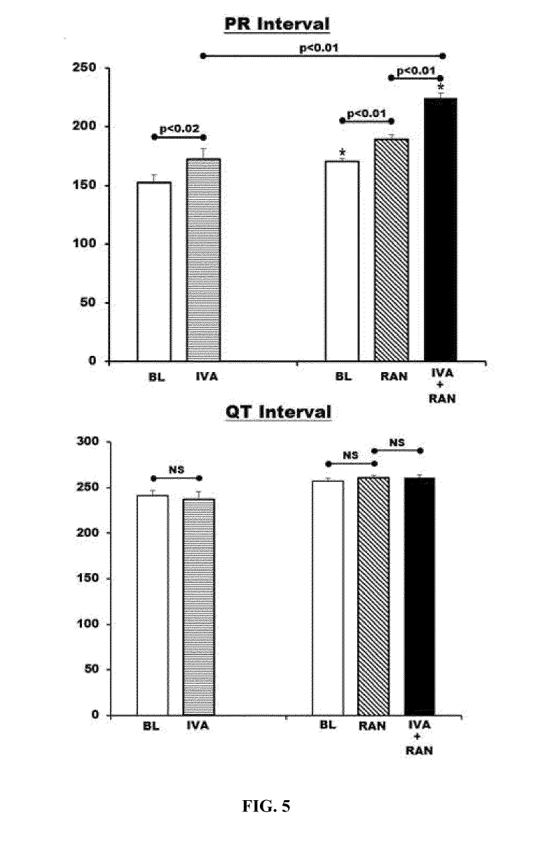

Influence of ivabradine (0.25 mg/kg) alone, ranolazine alone, and their combination on PR and QT intervals is illustrated in FIG. 5. Ivabradine (0.25 mg/kg) alone increased PR interval in a use-dependent manner. At pacing rates of 130 and 180 beats/min, the increase was 16±3.9 (p=0.05) and 20±3.5 msec (p<0.02), respectively, at 30 min after ivabradine administration. Ranolazine also increased PR interval in a use-dependent manner by 5±1.8 (NS) and by 19±2.8 msec (p<0.01), at pacing rates of 130 and 180 beats/min, respectively. Combined drug administration increased PR interval from baseline by 26±1.8 msec or 16%, and by 54±5.0 msec or 32%, both p<0.01, at pacing rates of 130 and 180 beats/min, respectively. These increases exceeded additive effects of both drugs alone (p<0.02 above ivabradine levels and p<0.01 above ranolazine levels during pacing at 130 beats/min; p<0.01 above levels achieved by both agents during pacing at 180 beats/min). The QT interval was not altered by either drug or by their combination at either pacing rate.

### 5. Effects of Ivabradine, Ranolazine and the Combination of Ivabradine and Ranolazine on A-H and H-V Intervals in Acetylcholine

### Mediated AF in Pigs

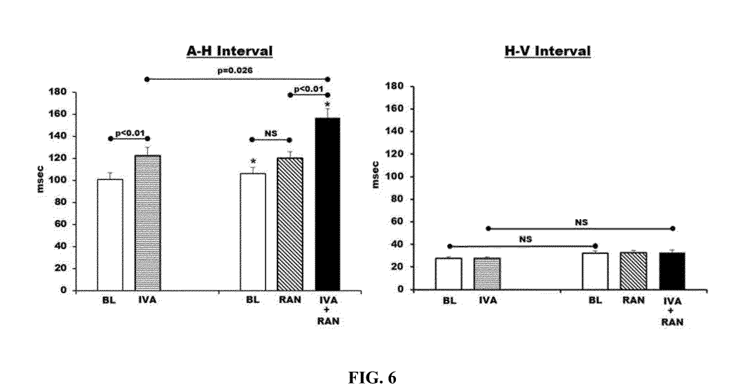

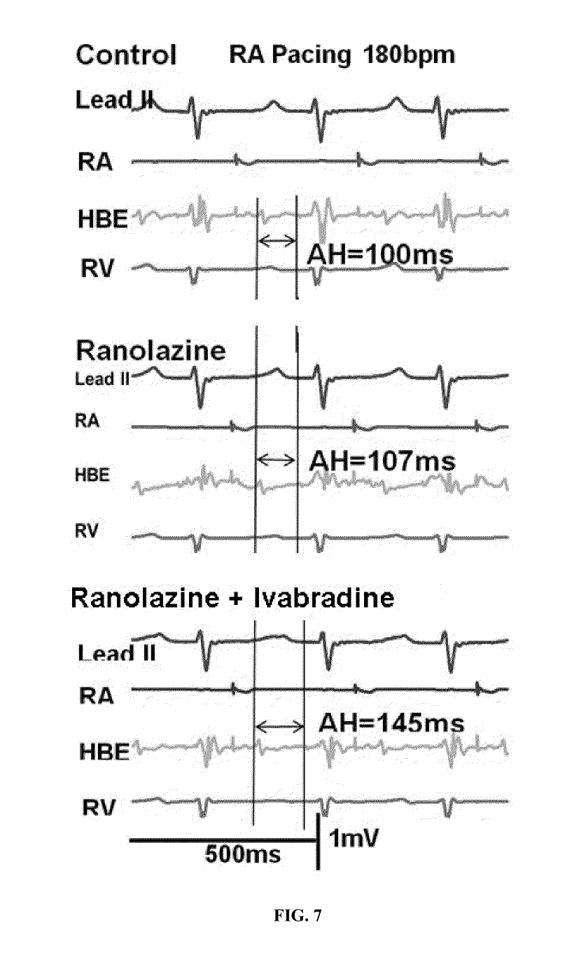

Ivabradine (0.25 mg/kg) alone also caused a marked use-dependent increase in A-H interval, as it increased A-H interval by 15.5 (NS) and by 22.0±3.5 msec (p<0.01), respectively, compared to control levels (FIG. 6) at atrial pacing rates of 130 and 180 beats/min. Ranolazine alone did not affect A-H interval during pacing at 130 beats/min (by 2.0±2.6 msec, NS) or at 180 beats/min (by 13.8±3.9 msec, NS). During pacing at 130 beats/min, the drug combination increased A-H interval by 20.8 msec or 20% (p<0.05 from baseline), a change that exceeded the additive effects of the agents alone. During pacing at 180 beats/min, the drug combination increased A-H interval by 50.5±6.2 msec or 48% (p<0.01 from baseline), a change that was greater than the additive effects of ivabradine alone (p<0.03) and ranolazine alone (p<0.01). Administration of the lower 0.1-mg/kg ivabradine dose after ranolazine increased AH interval by 14.0±5.5 msec (15%) and 28.7±9.6 msec (30%) at pacing rates of 130 and 180 beats/min, respectively, increases that were greater than the additive effects of the single agents. The H-V interval was unchanged at either pacing rate (FIG. 6). Recordings from a representative experiment are provided in FIG. 7.

### 6. Effects of Ivabradine, Ranolazine and the Combination of Ivabradine and Ranolazine on Dominant Frequency, Ventricular Rate, and Mean Arterial Blood Pressure During AF in Acetylcholine

### Mediated AF in Pigs

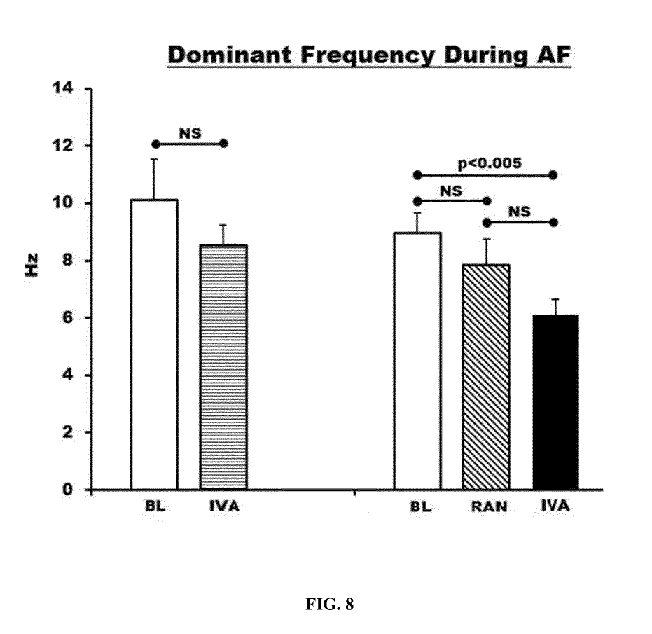

Neither ivabradine (0.25 mg/kg) alone (from baseline 10.3±1.4 to 9±0.7 Hz, NS) nor ranolazine alone (from baseline 9.0±0.8 to 7.8±0.9 Hz, NS) decreased dominant frequency of AF. However, the combination of agents significantly decreased dominant frequency (to 6.1±0.5, p=0.005) (FIG. 8). Likewise, the lower ivabradine dose (0.10 mg/kg) alone (from baseline 9.3±1.1 to 8.7, 1.1 Hz, NS) did not alter dominant frequency, but ranolazine alone (from baseline 8.5±1.1 to 6.7, 1.0 Hz, NS) did decrease this marker. Moreover, the decrease in dominant frequency of AF with the combination of the agents exceeded the additive effects of the single agents (to 5.7±0.8 Hz, p=0.02).

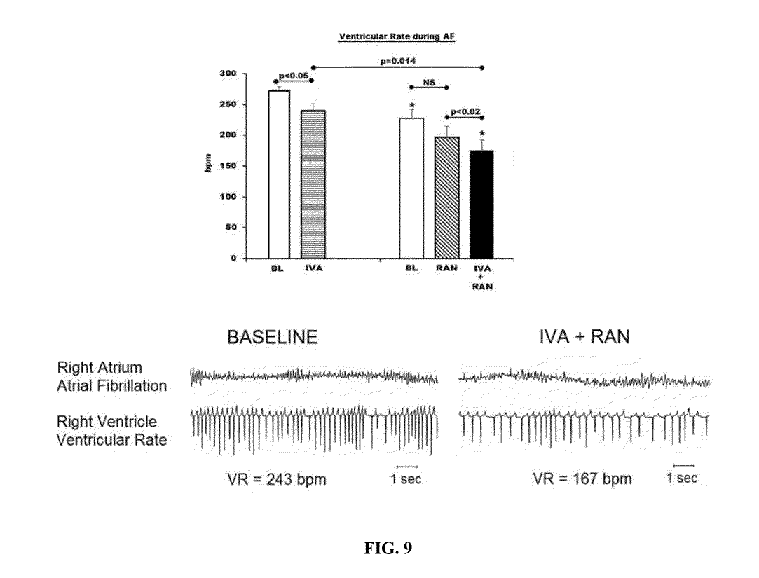

Ivabradine (0.25 mg/kg) alone reduced ventricular rate during AF by 22.5±6.0 beats/min or 9% (from 269.7±6.2 to 247.2±9.5 beats/min, p=0.015) at 30 min after drug infusion (FIG. 9, top panel), as illustrated in a representative experiment (FIG. 9, bottom panel). Ventricular rate was not reduced by ranolazine (from 227.3±14.7 to 197.1±17.4 beats/min, NS). But when ivabradine was administered shortly after ranolazine, ventricular rate was reduced by 51.9±9.7 or 23% (from 227.3±14.7 to 175.4±17.3 beats/min, p=0.02). Thus, the drug combination lowered ventricular rate (p<0.01) to a greater degree than either ivabradine (p<0.02) or ranolazine alone (p<0.02). Administration of the lower 0.1-mg/kg ivabradine dose after ranolazine reduced ventricular rate by 20% or 49.5±13.1 beats/min. This reduction was 27.0 beats/min (117%) greater than the 22.53±6.0-beats/min reduction achieved by 0.25 ivabradine alone and was similar to the 51.92±9.7-beats/min reduction achieved by ivabradine (0.25 mg/kg, i.v.) plus ranolazine.

Coadministration of the lower ivabradine dose (0.10 mg/kg) plus ranolazine also lowered ventricular rate during AF. Specifically, the effect of the combination is greater than that of ivabradine alone at the higher dose and is similar to that of the combination of ivabradine at the higher dose with ranolazine (data not shown).

This is the first demonstration in a large animal model of a significant reduction in ventricular rate during AF resulting from the combined actions of ivabradine and ranolazine. The synergistic effects of the agents were evident in prolongation of PR and A-H intervals. Consequently, ranolazine potentiated ivabradine's reduction in ventricular rate. The drugs did not alter QT or H-V intervals or change contractility. Although the plasma levels of ivabradine were 304±20.0 to 312±13.1 nM, which is higher than those obtained with the recommended chronic oral dose (7.5 mg/kg), the higher dose administered in the present study (0.25 mg/kg, i.v.) approximates the 0.20-mg/kg, i.v., dose demonstrated to be safe when tested in humans (Camm and Lau Drugs R D 2003; 4:83-9; Savelieva and Camm Drug Safety 2008; 31:95-107, Manz et al Cardiology 2003; 100:149-155.). The plasma level of ranolazine at the time of testing combined administration with ivabradine was 9.8±1.5 μM, which is not statistically different from the steady-state plasma therapeutic concentration of 2-8 μM (Reffelmann and Kloner Expert Rev Cardiovasc Ther 2010; 8:319-329, Verrier et al Heart Rhythm 2013; 10:1692-1697).

1. A method for controlling ventricular rate during atrial fibrillation or atrial flutter in a human patient in need thereof, comprising administering to the patient a therapeutically effective amount of ivabradine or a pharmaceutically acceptable salt thereof and a therapeutically effective amount of ranolazine.

2. A method for improving left ventricular function during atrial fibrillation or atrial flutter in a human patient in need thereof, comprising administering to the patient a therapeutically effective amount of ivabradine or a pharmaceutically acceptable salt thereof and a therapeutically effective amount of ranolazine.

3-13. (canceled)

14. The method of claim 1, wherein the ivabradine or a pharmaceutically acceptable salt thereof and the ranolazine are administered separately.

15. The method of claim 1, wherein the ivabradine or a pharmaceutically acceptable salt thereof and the ranolazine are administered intravenously.

16. The method of claim 1, wherein the ivabradine or a pharmaceutically acceptable salt thereof and the ranolazine are administered orally.

17. The method of claim 16, wherein the ivabradine or a pharmaceutically acceptable salt thereof and the ranolazine are administered as a combined dosage unit.

18. The method of claim 17, wherein the combined dosage unit is a tablet.

19. The method of claim 18, wherein the amount of the ranolazine administered is from about 50 mg to about 3000 mg daily.

20. The method of claim 19, wherein the amount of the ranolazine administered is from about 50 mg to about 1500 mg daily.

21. The method of claim 16, wherein the ranolazine is administered as a sustained release formulation.

22. The method of claim 16, wherein the amount of the ivabradine or a pharmaceutically acceptable salt thereof administered is from about 1 mg to about 50 mg daily.

23. The method of claim 16, wherein the amount of the ivabradine or a pharmaceutically acceptable salt thereof administered is from about 1 mg to about 10 mg daily.

24. The method of claim 16, wherein the amount of the ivabradine or a pharmaceutically acceptable salt thereof administered is about 1 mg, about 5 mg, or about 10 mg daily.

25. The method of claim 15, wherein the amount of the ranolazine administered is from about 10 mg/hr to about 200 mg/hr.

26. The method of claim 15, wherein the amount of the ranolazine administered is from about 10 mg/hr to about 100 mg/hr.

27. The method of claim 15, wherein the amount of the ranolazine administered is from about 10 mg/hr to about 50 mg/hr.

28. The method of claim 15, wherein the ivabradine or a pharmaceutically acceptable salt thereof is administered as a bolus injection.

29. The method of claim 28, wherein the amount of the ivabradine or a pharmaceutically acceptable salt thereof administered is from about 0.01 mg/kg to about 1 mg/kg.

30. The method of claim 29, wherein the amount of the ivabradine or a pharmaceutically acceptable salt thereof administered is from about 0.01 mg/kg to about 0.25 mg/kg.

31. The method of claim 29, wherein the amount of the ivabradine or a pharmaceutically acceptable salt thereof administered is from about 0.01 mg/kg to 0.1 mg/kg.

32. The method of claim 16, wherein the ivabradine or a pharmaceutically acceptable salt thereof is administered as an immediate release or sustained release formulation.

33. The method of claim 16, wherein the ivabradine or a pharmaceutically acceptable salt thereof and/or the ranolazine are administered twice daily.

34. The method of claim 16, wherein the ivabradine or a pharmaceutically acceptable salt thereof and/or the ranolazine are administered once daily.

35. The method of claim 6, wherein A-H interval is increased when atrial rate is high.

36. The method of claim 35, wherein the patient suffers from atrial fibrillation.

37. The method of claim 8, wherein AV conduction is slowed when atrial rate is high.

38. The method of claim 8, wherein atrial rate is slowed.

39. The method of claim 8, wherein the patient suffers from atrial fibrillation.

40. The method of claim 10, wherein the patient suffers from atrial fibrillation.

41. The method of claim 1, wherein the arterial blood pressure is maintained or increased in the human patient.

42. The method of claim 41, wherein the arterial blood pressure is increased.

43. The method of claim 1, wherein the amount of the ivabradine or a pharmaceutically acceptable salt thereof administered is from about 5 mg to about 15 mg daily, and the amount of the ranolazine administered is from about 300 mg to about 1000 mg daily.

44. The method of claim 1, wherein the ivabradine is ivabradine hydrochloride.

45. A pharmaceutical formulation comprising a therapeutically effective amount of ivabradine or a pharmaceutically acceptable salt thereof and a therapeutically effective amount of ranolazine, and a pharmaceutically acceptable carrier.

46. The pharmaceutical formulation of claim 45, formulated for intravenous administration.

47. The pharmaceutical formulation of claim 46, formulated for oral administration.

48. The pharmaceutical formulation of claim 47, wherein the formulation is in tablet form or capsule form.

49. The pharmaceutical formulation of claim 48, wherein the tablet or capsule comprises from about 1 mg to about 20 mg of the ivabradine or the pharmaceutically acceptable salt thereof.

50. The pharmaceutical formulation of claim 48, wherein the tablet or capsule comprises from about 2.5 mg to about of 7.5 mg of the ivabradine or the pharmaceutically acceptable salt thereof.

51. The pharmaceutical formulation of claim 48, wherein the tablet or capsule comprises about 2.5 mg, about 5 mg, or about 7.5 mg of the ivabradine or the pharmaceutically acceptable salt thereof.

52. The pharmaceutical formulation of claim 48, wherein the tablet or capsule comprises from about 50 mg to about 1000 mg of the ranolazine.

53. The pharmaceutical formulation of claim 48, wherein the tablet or capsule comprises from about 100 mg to about 750 mg of the ranolazine.

54. The pharmaceutical formulation of claim 48, wherein the tablet or capsule comprises from about 150 mg to about 375 mg of the ranolazine.

55. The pharmaceutical formulation of claim 48, wherein the ranolazine is formulated for sustained release.

56. The pharmaceutical formulation of claim 48, wherein the ivabradine or the pharmaceutically acceptable salt thereof is formulated for immediate release.

57. The pharmaceutical formulation of claim 48, wherein the ivabradine or the pharmaceutically acceptable salt thereof is formulated for sustained release.

58. The pharmaceutical formulation of claim 45, wherein the pharmaceutically acceptable salt of ivabradine is ivabradine hydrochloride.

59-60. (canceled)
